(19) United States
(12) Patent Application Publication
(10) Pub. No.: US 2023/0022457 A1
(43) Pub. Date: Jan. 26, 2023

(54) METHOD FOR PRODUCING POWDER SOLID COMPOSITION AND POWDER SOLID COMPOSITION

(71) Applicant: SHISEIDO COMPANY, LTD., Tokyo (JP)

(72) Inventors: Hiroto MUKOYAMA, Tokyo (JP); Ken HIROSAKI, Tokyo (JP); Kiyoshi TAKAHASHI, Tokyo (JP)

(21) Appl. No.: 17/757,793

(22) PCT Filed: Dec. 14, 2020
(86) PCT No.: PCT/JP2020/046534

§ 371 Date: Jun. 21, 2022

(30) Foreign Application Priority Data
Dec. 25, 2019 (JP) 2019-234724

(51) Int. Cl. A61K 8/02; A61Q 1/12; A61K 8/26; A61K 8/891; A61K 8/92; A61K 8/29

(52) U.S. Cl. CPC A61K 8/022; A61Q 1/12; A61K 8/26; A61K 8/891; A61K 8/92; A61K 8/29; A61K 2800/43

(57) ABSTRACT

A method for producing a powder solid composition including a base containing non-volatile liquid oil, the method includes: blending wax in a powder form into the base; dispersing the base in a solvent; volatilizing the solvent at a first temperature where the wax does not dissolve; and heating the base at a second temperature where the wax dissolves, wherein a blending amount of the non-volatile liquid oil is 15% by mass or more and 40% by mass or less with respect to the base.

### TECHNICAL FIELD

The present invention relates to a method for producing a powder solid composition and relates to a powder solid composition.

### BACKGROUND ART

Powder cosmetics such as foundations and eye shadows contain oil in order to give a feeling such as a moist feeling and gloss when used. In conventional powder cosmetics, wax is blended to provide impact resistance (see, for example, PTL 1). In addition, conventional powder cosmetics contain ingredients such as a luster material to give additional properties such as pearlescence when used (see, for example, PTL 2).

### CITATION LIST

### Patent Literature

[PTL 1]

Japanese Laid-Open Patent Publication No. 2005-220053
[PTL 2]

Japanese Laid-Open Patent Publication No. 2002-275033

### SUMMARY OF INVENTION

### Technical Problem

In conventional powder cosmetics, when a blending amount of ingredients such as a luster material is increased or particle size of the ingredients is increased, impact resistance will decrease. Therefore, there is a limit to improving additional properties such as pearlescence. In addition, when dissolving wax during manufacture of the powder cosmetics, film of the dissolved wax may prevent volatilization of solvent, which may reduce a feeling when used of the resulting powder cosmetics, although it is possible to suppress reduction in impact resistance to some extent.

It is an object of the present invention to provide a method for producing a powder solid composition that is capable of improving additional properties without deteriorating a feeling when used.

### Solution to Problem

One aspect of the present invention is: a method for producing a powder solid composition including a base containing non-volatile liquid oil, the method including blending wax in a powder form into the base; dispersing the base in a solvent; volatilizing the solvent at a first temperature where the wax does not dissolve; and heating the base at a second temperature where the wax dissolves, wherein a blending amount of the non-volatile liquid oil is 15% by mass or more and 40% by mass or less with respect to the base.

### Advantageous Effects of Invention

According to one aspect of the present invention, a method for producing a powder solid composition that is capable of improving additional properties without deteriorating a feeling when used, can be provided.

### BRIEF DESCRIPTION OF THE DRAWINGS

FIG. 1 is a flowchart of a method for producing a powder solid composition according to an embodiment of the present invention;

FIG. 2 is a diagram illustrating a step of a method for producing (a method for blending) a powder solid composition according to the present embodiment; and

FIG. 3 is a diagram illustrating a step of a method for producing (a method for molding) a powder solid composition according to the present embodiment.

### DESCRIPTION OF EMBODIMENTS

Embodiments of the present invention will be described in detail with reference to the drawings. In some cases, the parts common to each drawing may be designated by the same reference numerals and description thereof may be omitted.

### (Method for Producing Powder Solid Composition)

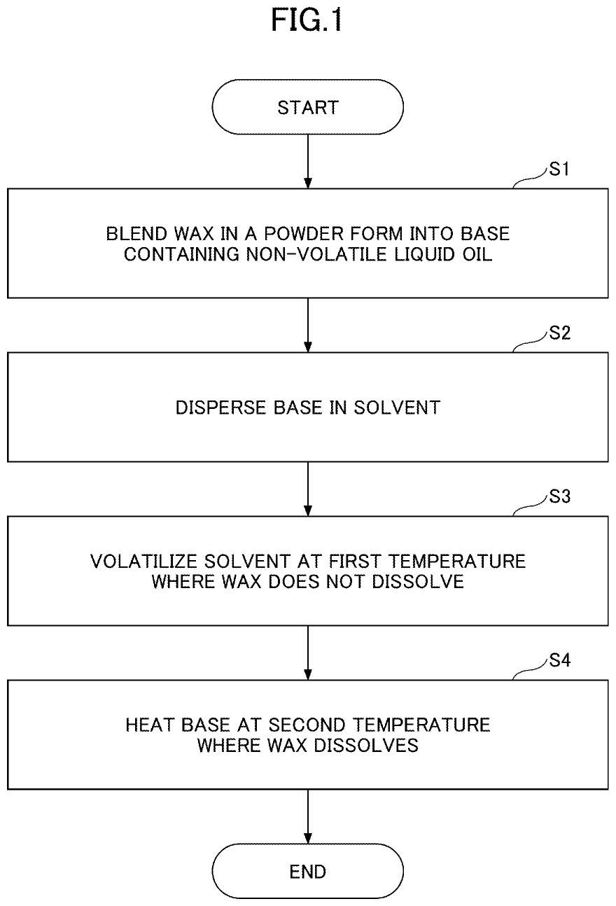

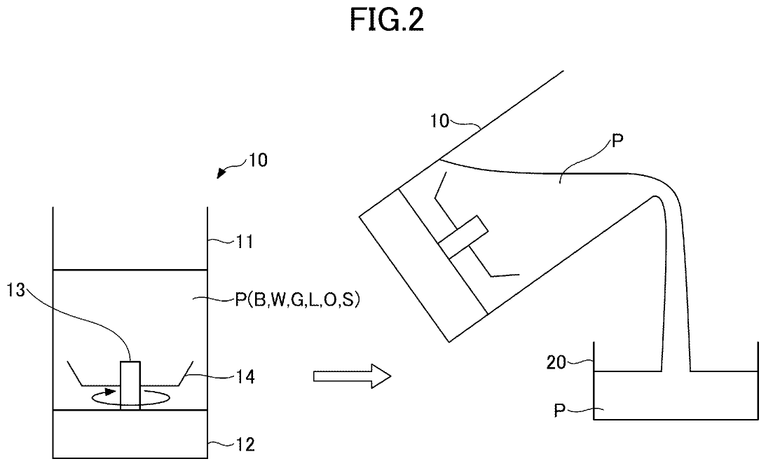

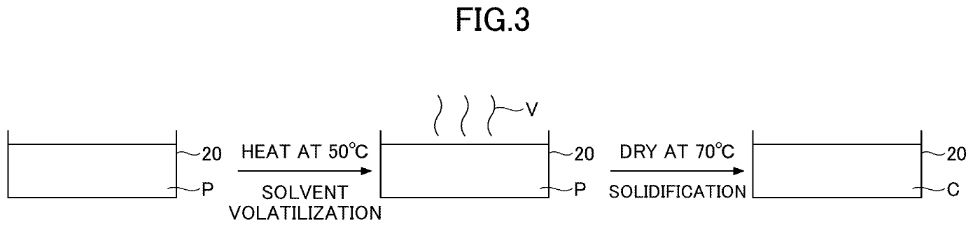

FIG. 1 is a flowchart of a method for producing a powder solid composition according to an embodiment of the present invention. FIGS. 2 and 3 are flowcharts of a method for producing a powder solid composition according to the present embodiment.

The method for producing a powder solid composition according to the present embodiment is a method for producing a powder solid composition including a base containing non-volatile liquid oil. As used herein, a powder solid composition refers to a powder or a composition containing a powder formed into a solid. The base may include a powder or a composition containing a powder as a main ingredient. The liquid oil refers to an oil component or oil or fat that is in a liquid state. “Non-volatile” means that the kinematic viscosity is 5 cSt (centistokes) or more.

The method for producing a powder solid composition according to the present embodiment includes a step of blending wax in a powder form into a base containing non-volatile liquid oil (see step S1 of FIG. 1). Specifically, powder B, wax W, non-volatile liquid oil L, luster powder G, and non-volatile silicone oil O are added to a Henschel mixer 10 and kneaded (see FIG. 2).

The configuration of the Henschel mixer 10 is freely determined, and may include, for example, a mixing vessel 11, a main unit 12 having a built-in motor, a rotating shaft 13, and a blade 14 (see FIG. 2). The mode of kneading the powder B, the wax W, and the like is not limited thereto.

Ingredients of the powder are not particularly limited, and may be, for example, talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, tungstate, magnesium, silica, zeolite, barium sulfate, calcined calcium sulfate (calcined gypsum), calcium phosphate, fluorine apatite, hydroxyapatite, ceramic powder, silicone powder, magnesium myristate, zinc myristate, calcium palmitate, aluminum stearate, chlorphenesin, boron nitride, and the like. These ingredients of the powder may be used alone or in a mixture of two or more.

In the method for producing a powder solid composition according to the present embodiment, the blending amount of the powder is freely determined, but is preferably 10% by mass or more and 70% by mass or less, more preferably 15% by mass or more and 65% by mass or less, and even more preferably 20% by mass or more and 60% by mass or less, with respect to 100% by mass of the powder solid composition.

The wax W in the Henschel mixer 10 is kneaded in a pulverized state. The pulverized wax is an example of the wax in a powder form in the method for producing a powder solid composition according to the present invention. The wax is not particularly limited, and preferably wax having a melting point of 55° C. or more and 90° C. or less, more preferably wax having a melting point of 60° C. or more and 85° C. or less, and even more preferably wax having a melting point of 65° C. or more and 80° C. or less.

Ingredients of the wax are not particularly limited, and may be, for example, beeswax, candelilla wax, cotton wax, carnauba wax, bayberry wax, insect wax, spermaceti, montan wax, bran wax (rice wax), kapok wax, Japan wax, lanolin acetate, liquid lanolin, sugarcane wax, lanolin fatty acid isopropyl ester, hexyl laurate, reduced lanolin, jojoba ester (hydrogenated jojoba oil), hardened lanolin, shellac wax, beeswax, microcrystalline wax, paraffin wax, POE lanolin alcohol ether, POE lanolin alcohol acetate, POE cholesterol ether, lanolin fatty acid polyethylene glycol, fatty acid glyceride, hydrogenated castor oil, POE hydrogenated lanolin alcohol ether, sucrose fatty acid ester (sucrose tetrastearate triacetate), and the like. These ingredients of the wax may be used alone or in a mixture of two or more.

The blending amount of the wax is freely determined, but may be 0.5% by mass or more and 10% by mass or less, preferably 1% by mass or more and 6.5% by mass or less, more preferably 1.5% by mass or more and 6% by mass or less, and even more preferably 2% by mass or more and 5.5% by mass or less, with respect to the base.

Ingredients of the non-volatile liquid oil are not particularly limited, and may be, for example, liquid paraffin, ozocerite, squalane, pristane, ceresin, vaseline, glyceryl tri-2-ethylhexanoate, isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethylol propane tri-2-ethyl hexanoate, trimethylol propane triisostearate, pentaerythritol tetra-2-ethyl hexanoate, glyceryl tri-2-ethyl hexanoate, trimethylol propane triisostearate, cetyl 2-ethylhexanoate, 2-ethylhexylpalmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, triethyl citrate, crotamiton (C₁₃H₁₇NO) , and the like. These ingredients of the liquid oil may be used alone or in a mixture of two or more.

The blending amount of the non-volatile liquid oil is freely determined, but is preferably 15% by mass or more and 40% by mass or less, more preferably 20% by mass or more and 38% by mass or less, and even more preferably 25% by mass or more and 35% by mass or less, with respect to the base.

The luster powder may be freely blended. In the present embodiment, the luster powder G in the Henschel mixer 10 is kneaded together with the powder B and the wax W in a pulverized state. The pulverized luster powder G is an example of the luster powder in the method for producing a powder solid composition of the present invention.

The average particle size of the luster powder is freely determined, but is preferably 30 μm or more, more preferably 35 μm or more, and even more preferably 40 μm or more. The average particle size is a particle size in which the cumulative distribution on a volume basis measured by the laser diffraction/scattering method is 50%.

The upper limit of the average particle size of the luster powder is not particularly limited. From the viewpoint of maintaining impact resistance of the powder solid composition, the average particle size of the luster powder is preferably 200 μm or less, more preferably 150 μm or less, and even more preferably 100 μm or less.

Ingredients of the luster powder are not particularly limited, and may be, for example, titanium oxide coated mica, titanium oxide coated synthetic mica, titanium oxide coated silica, titanium oxide coated talc, zinc oxide coated silica, titanium oxide coated colored mica, colcothar coated mica titanium, colcothar and black iron oxide coated mica titanium, carmine coated mica titanium, and iron blue coated mica titanium. These ingredients of the luster powder may be used alone or in a mixture of two or more.

The blending amount of the luster powder is freely determined, but is preferably 10% by mass or more and 60% by mass or less, more preferably 15% by mass or more and 55% by mass or less, and even more preferably 20% by mass or more and 50% by mass or less, with respect to the base.

The non-volatile silicone oil may be freely blended. The non-volatile silicone oil refers to a silicone oil having a kinematic viscosity of 5 cSt (centistokes) or more. Ingredients of the non-volatile silicone oil are not particularly limited, and may be, for example, dimethyl polysiloxane (dimethicone), methylphenyl polysiloxane, methylhydrogen polysiloxane, and the like. These ingredients of the silicone oil may be used alone or in a mixture of two or more.

In the method for producing a powder solid composition according to the present embodiment, the blending amount of the non-volatile silicone oil is freely determined, but is preferably 10% by mass or more and 30% by mass or less, more preferably 13% by mass or more and 28% by mass or less, and more preferably 15% by mass or more and 25% by mass or less, with respect to the base.

The method for producing a powder solid composition according to the present embodiment includes a step of dispersing the base in a solvent (see step S2 of FIG. 1). “Dispersing the base in a solvent” refers to producing the powder solid composition in a wet process. Specifically, a solvent S is further added in the Henschel mixer 10 and stirred (see FIG. 2).

Ingredients of the solvent is not particularly limited, and may be, for example, water, ethanol, propanol, isopropanol, isobutyl alcohol, t-butyl alcohol, and the like. These ingredients of the solvent may be used alone or in a mixture of two or more.

The blending amount of the solvent compounded is freely determined, but preferably 3% by mass or more and 50% by mass or less, more preferably 5% by mass or more and 40% by mass or less, and more preferably 10% by mass or more and 30% by mass or less, with respect to 100% by mass of the base (total amount of the base containing the wax, the luster powder, and the like).

In the method for producing a powder solid composition according to the present embodiment, a coloring agent, an antioxidant, a preservative, and the like, other than the ingredients described above may be added to the extent that the effect of the present invention is not impaired.

Examples of the coloring agent include red iron oxide, yellow iron oxide, black iron oxide, inorganic white pigments (for example, zinc oxide, and the like); inorganic red pigments (for example, iron titanate, and the like); inorganic violet pigments (for example, mango violet, cobalt violet, and the like); inorganic green pigments (for example, chromium oxide, chromium hydroxide, cobalt titanate, and the like); inorganic blue pigments (for example, ultramarine, iron blue, and the like); metal powder pigments (for example, aluminum powder, copper powder, and the like); organic pigments such as zirconium, barium, or aluminum lake (for example, organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404; Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, Blue No. 1, and the like); natural dyes (for example, chlorophyll, β-carotene, and the like) and the like. The blending amount of the coloring agent is freely determined.

Examples of the antioxidant include ascorbic acid, alpha-tocopherol, dibutyl hydroxytoluene, butyl hydroxyanisole, and the like.

The blending amount of the antioxidant is freely determined, but preferably 0.005% by mass or more and 0.2% by mass or less, more preferably 0.05% by mass or more and 0.1% by mass or less, and more preferably 0.015% by mass or more and 0.05% by mass or less.

Examples of the preservative include paraoxybenzoic acid ester, phenoxyethanol, octoxyglycerin, benzoic acid, salicylic acid, carbolic acid, sorbic acid, parachloromethacresol, hexachlorophene, benzalkonium chloride, chlorhexidine chloride, trichlorocarbanilide, and the like.

The blending amount of the preservative is freely determined, but preferably 0.05% by mass or more and 0.5% by mass or less, more preferably 0.1% by mass or more and 0.4% by mass or less, and more preferably 0.15% by mass or more and 0.3% by mass or less.

The powder B, the wax W, the luster powder G, the non-volatile liquid oil L, the non-volatile silicone oil O, and the solvent S in the Henschel mixer 10 are stirred to form a paste slurry P (see FIG. 2). Then, the slurry P in the Henschel mixer 10 is poured into a mold 20 (see FIG. 2). Preferably, the slurry P is poured into the mold 20 while being filtered.

The method for producing a powder solid composition according to the present embodiment includes a step of volatilizing the solvent at a first temperature where the wax does not dissolve (see step S3 of FIG. 1). The first temperature is freely determined, but is preferably lower than the melting point of the wax (the temperature at which the wax does not dissolve). “Volatilizing the solvent” refers to vaporization of the liquid solvent.

In this step, the slurry P filled in the mold 20 is heated at a temperature (about 50° C.) lower than the melting point of the wax (see FIG. 3). The mode of heating the slurry P is freely determined, and for example, the mold 20 filled with the slurry

P may be placed in a thermostatic bath equipped with a degassing device and dried. The heating time of the slurry P is freely determined, and is, for example about 3 minutes to 3 hours.

As a result, the solvent S in the slurry P volatilizes and is removed and degassed from the slurry P as a volatile solvent V. The wax W in the slurry P does not dissolve and remains in the slurry P as a powder.

The method for producing a powder solid composition according to the present embodiment includes a step of heating the base at a second temperature where the wax dissolves (see step S4 of FIG. 1). The second temperature is freely determined, but is preferably equal to or higher than the melting point of the wax (the temperature at which the wax melts). “Heating the base” refers to applying heat to the base to dry the base.

Specifically, the second temperature is preferably 55° C. or more and 90° C. or less, more preferably 60° C. or more and 85° C. or less, and more preferably 65° C. or more and 80° C. or less.

In this step, the slurry P filled in the mold 20 is dried at a temperature (about 70° C.) equal to or higher than the melting point of the wax (see FIG. 3). The mode of heating the base is freely determined, and for example, the mold 20 filled with the slurry P may be continuously placed in the thermostatic bath described above, and dried by raising the temperature in the thermostatic bath. The heating time of the base is freely determined, and for example, about 3 minutes to 3 hours.

As a result, the slurry P is dried with the solvent S removed, and the wax W in a powder form dissolves in the slurry P. The dried base is then cooled to room temperature to obtain a solid.

The method for producing a powder solid composition according to the present embodiment is suitably used in production of solid powder cosmetics such as foundations, eye shadows, cheek colors, body powders, perfume powders, baby powders, pressed powders, deodorant powders, face powders, and the like.

In the present embodiment, as described above, by blending wax in a powder form into the base containing the non-volatile liquid oil and volatilizing the solvent dispersed in the base at the first temperature where the wax does not dissolve, the powder form of the wax blended in the base is maintained when the solvent volatilizes. Therefore, the wax blended in the base does not interfere with the volatilization of the solvent.

By heating the base containing the wax at the second temperature where the wax dissolves, the wax in a powder form can be dissolved in a state where the solvent is removed. The powder solid composition obtained by solidifying the dissolved wax at room temperature is highly impact resistant. Therefore, even when ingredients such as luster powder is blended in the base, it is possible to prevent a decrease in impact resistance of the powder solid composition.

By dissolving the wax in a powder form in a state where the solvent is removed, the residual solvent in the powder solid composition is reduced. Accordingly, a feeling (for example, a moist feeling, gloss, softness, easiness to take, and the like) when the resulting powder solid composition is used is improved. Thus, according to the present embodiment, the additional properties of the powder solid composition is improved without deteriorating a feeling when used.

Further, by setting the blending amount of the non-volatile liquid oil to 15% by mass or more and 40% by mass or less with respect to the base, a feeling when the resulting powder solid composition is used is improved. Therefore, according to the present embodiment, a powder solid composition that imparts an excellent feeling when used can be obtained.

In the present embodiment, as described above, when the first temperature is a temperature lower than the melting point of the wax, the first temperature for volatilizing the solvent can be set to a temperature at which the wax does not dissolve. Thus, the powder form of the wax blended in the base is maintained when the solvent volatilizes. Therefore, the wax does not interfere with the volatilization of the solvent.

In the present embodiment, as described above, when the second temperature is a temperature equal to or higher than the melting point of the wax, the second temperature for heating the base can be set to a temperature at which the wax dissolves. Thus, the wax in a powder form can be dissolved in a state where the solvent is removed. Therefore, it is possible to prevent a decrease in impact resistance of the resulting powder solid composition, and to improve a feeling when the resulting powder solid composition is used.

In the present embodiment, as described above, when the second temperature range is 55° C. or more and 90° C. or less, the temperature range of the second temperature at which the wax dissolves does not overlap the temperature range of the first temperature at which the solvent volatilizes and the wax does not dissolve. Therefore, the wax in a powder form can be dissolved in a state where the solvent is removed.

In the present embodiment, when the second temperature is 55° C. or more and 90° C. or less, wax having a melting point of 55° C. or more and 90° C. or less can be used as the wax. All waxes having a melting point of 55° C. or more and 90° C. or less are solid at room temperature and are easy to handle. When wax having a melting point of 55° C. or more and 90° C. or less is used, there is a wide range of choices for the type of wax.

In the present embodiment, as described above, when the non-volatile silicone oil is blended in the non-volatile liquid oil, a feeling when the resulting powder solid composition is used is further improved. Thus, according to the present embodiment, a powder solid composition that imparts an even better feeling when used can be obtained.

As described above, when the blending amount of the non-volatile silicone oil is 10% by mass or more and 30% by mass or less with respect to the base, a feeling when the resulting powder solid composition is used is further improved. Thus, according to the present embodiment, a powder solid composition that imparts an even better feeling when used can be obtained.

In the present embodiment, as described above, when the wax having a melting point of 55° C. or more and 90° C. or less is used as the wax in a powder form, the solvent can be volatilized without dissolving the wax at the first temperature, and the wax can be dissolved at the second temperature. Thus, the wax does not interfere with the volatilization of the solvent. Therefore, the wax in a powder form can be dissolved in a state where the solvent is removed.

In the present embodiment, as described above, when the blending amount of the wax is 0.5%. by mass or more and 10% by mass or less with respect to the base, impact resistance of the resulting powder solid composition is improved. In addition, when the blending amount of the wax is in such a range, a feeling when the resulting powder solid composition is used is improved.

In the present embodiment, as described above, because impact resistance of the powder solid composition is improved, even when the luster powder is blended in the base, it is possible to prevent a decrease in impact resistance of the powder solid composition. Therefore, in the present embodiment, by blending the luster powder in the base, pearlescence is improved without deteriorating a feeling of the resulting powder solid composition when used.

In addition, because impact resistance of the powder solid composition is improved, even when the blending amount of the luster powder is increased, it is possible to prevent a decrease in impact resistance of the powder solid composition. Thus, in the present embodiment, by blending the luster powder of 10% by mass or more and 60% by mass or less with respect to the base, pearlescence of the resulting powder solid composition is further improved.

In the present embodiment, as described above, because impact resistance of the powder solid composition is improved, even when the particle size of the luster powder blended in the base is increased, it is possible to prevent a decrease in impact resistance of the powder solid composition. Thus, in the present embodiment, by blending the luster powder having an average particle size of 30 μm or more, pearlescence is improved with high accuracy without deteriorating a feeling of the resulting powder solid composition when used.

### <Powder Solid Composition>

The powder solid composition according to the present embodiment includes non-volatile liquid oil containing non-volatile silicone oil, wax having a melting point of 55° C. or more and 90° C. or less, and luster powder having an average particle size of 30 μm or more.

The blending amount of the non-volatile liquid oil is 15% by mass or more and 40% by mass or less, preferably 23% by mass or more and 38% by mass or less, and more preferably 25% by mass or more and 35% by mass or less, with respect to 100% by mass of the powder solid composition.

The blending amount of the non-volatile silicone oil is freely determined, but preferably 10% by mass or more and 30% by mass or less, more preferably 13% by mass or more and 28% by mass or less, and even more preferably 15% by mass or more and 25% by mass or less, with respect to 100% by mass of the powder solid composition.

blending amount of the wax having a melting point of 55° C. or more and 90° C. or less is 2% by mass or more and 10% by mass or less, preferably 2.5% by mass or more and 4.5% by mass or less, and 3% by mass or more and 4% by mass or less, with respect to 100% by mass of the powder solid composition.

The melting point of the wax is preferably 60° C. or more and 85° C. or less, and more preferably 65° C. or more and 80° C. or less. The form of the wax is not particularly limited, and is preferably a powder form.

The blending amount of the luster powder having an average particle size of 30 μm or more is 10% by mass or more and 60% by mass or less, preferably 15% by mass or more and 55% by mass or less, and more preferably 20% by mass or more and 50% by mass or less, with respect to 100% by mass of the powder solid composition.

The average particle size of the luster powder is preferably 30 μm or more, more preferably 35 μm or more, and even more preferably 40 μm or more. The upper limit of the average particle size of the luster powder is freely determined. From the viewpoint of maintaining impact resistance of the powder solid composition, the average particle size of the luster powder is preferably 200 μm or less, more preferably 150 μm or less, and even more preferably 100 μm or less.

The powder solid composition according to the present embodiment is obtained by the method for producing a powder solid composition described above. That is, the powder solid composition according to the present embodiment is obtained by performing the steps of: blending wax in a powder form into a base containing non-volatile liquid oil; dispersing the base in a solvent; volatilizing the solvent at a first temperature where the wax does not dissolve; and heating the base at a second temperature where the wax dissolves.

The powder solid composition according to the present embodiment is suitably used in solid powder cosmetics such as foundations, eye shadows, cheek colors, body powders, perfume powders, baby powders, pressed powders, deodorant powders, face powders, and the like.

The powder solid composition thus obtained has high impact resistance and provides an excellent feeling when used. In the present embodiment, as described above, the non-volatile liquid oil containing the non-volatile silicone oil, the wax having a melting point of 55° C. or more and 90° C. or less, and the luster powder having an average particle size of 30 μm or more, are included within respective predetermined ranges. Accordingly, pearlescence is improved while maintaining the feeling of the powder solid composition when used.

In the powder solid composition according to the present embodiment, the wax in a powder form does not dissolve in the step of volatilizing the solvent at the first temperature, and dissolves in the step of heating the base at the second temperature. In the resulting powder solid composition, the solvent has been removed and the powder form of the wax has disappeared. Therefore, at present, it is technically difficult to specify and characterize the structure or characteristics of the powder solid composition according to the present embodiment by analyzing the structure or characteristics of the powder solid composition in which the wax in a powder form is dispersed in the solvent together with the base, the non-volatile liquid oil, the luster powder, and the like, before the production of the powder solid composition.

In addition, even when the structure or characteristics of the powder solid composition according to the present embodiment can be analyzed by some method, it would be extremely expensive and time consuming to perform the work of specifying the structure or characteristics of the powder solid composition according to the present embodiment, in view of the fact that speed and the like are required due to the nature of the patent application. That is, there are impossible and impractical circumstances for directly specifying the powder solid composition according to the present embodiment by specifying its structure or characteristics.

### EXAMPLE

Hereinafter, the present invention will be further described with reference to examples. In the following, “parts” and “%” are based on mass unless otherwise specified. Various tests and evaluations were performed according to the following methods.

### <Test Sample>

A base is kneaded by adding powder, wax, a coloring agent, luster powder, non-volatile liquid oil, and non-volatile silicone oil into a Henschell mixer (manufactured by Kyoritsu Riko Corporation, SK-M) (see step Sl in FIG. 1, and FIG. 2). A solvent is then added to the Henschel mixer and stirred with a mixer (manufactured by PRIMIX Corporation, ROBOMIX) to produce a slurry (a base in a wet process) P (see step S2 in FIG. 1, and FIG. 2). The slurry P in the Henschel mixer is poured into a mold 20 in a plate shape (see FIG. 2). The mold 20 filled with the slurry P is placed in a thermostatic bath (manufactured by Yamato Scientific Co., Ltd., DF611) and heated at about 50° C. (a temperature lower than the melting point of the wax) for about 3 hours (see step S3 in FIG. 1, and FIG. 3). Thereafter, the temperature in the thermostatic bath is adjusted to about 70° C. (a temperature equal to or higher than the melting point of the wax) and further heated (dried) for about 1 hour (see step S4 in FIG. 1, and FIG. 3). The mold 20 is removed from the thermostatic bath and is left in the room for about 1 hour. The resulting compact (solid) C is used as a test sample (see FIG. 3). The method for producing the test sample is not limited to the conditions described above, and can be appropriately changed according to the producing equipment of the factory and the like and the producing scale.

### <Impact Resistance>

The test sample was dropped from a height of 30 cm onto a metal plate. The number of times until the test sample cracked was examined. For each of the test samples, it was evaluated as good when the average value of the tests (N=3) was 5 times or more.

### <Moist Feeling>

A panel of two experts evaluated a moist feeling when the test sample was applied around the eyes (eyelids) according to the following criteria. When the score was 2 or more, the moist feeling was evaluated as good.
5: Feels very moist
4: Feels moist
3: Feels somewhat moist
2: Feels slightly moist
1: Does not feel moist at all

### <Gloss>

A panel of two experts evaluated gloss when the test sample was applied around the eyes (eyelids) according to the following criteria. When the score was 2 or more, the gloss was evaluated as good.
5: Feels very glossy
4: Feels glossy
3: Feels somewhat glossy
2: Feels slightly glossy
1: Does not feel glossy at all

### <Softness>

A panel of two experts evaluated softness when the test sample was applied around the eyes (eyelids) according to the following criteria. When the score was 2 or more, the softness was evaluated as good.
5: Feels very soft
4: Feels soft
3: Feels somewhat soft
2: Feels slightly soft
1: Does not feel soft at all

### <Pearlescence>

A panel of two experts evaluated pearlescence when the test sample was applied around the eyes (eyelids) according to the following criteria. When the score was 2 or more, the pearlescence was evaluated as good.
5: Looks very pearly
4: Looks pearly
3: Looks somewhat pearly
2: Looks slightly pearly
1: Does not look pearly at all

### <Easiness to Take>

A panel of two experts evaluated easiness to take when the test sample was applied around the eyes (eyelids) according to the following criteria. When the score was 2 or more, the easiness to take was evaluated as good.
5: Feels very easy to take
4: Feels easy to take
3: Feels somewhat easy to take
2: Feels slightly easy to take
1: Does not feel easy to take at all

Hereinafter, examples and comparative examples will be described.

### Example 1

To make a total of 100 parts of the base, 2.5 parts of talc, 10 parts of synthetic phlogopite, 1 part of zinc oxide, 1 part of magnesium myristate, 4 parts of (vinyl dimethicone/methicone silsesquioxane) crosspolymer, and 8 parts of boron nitride were mixed as the powder, 2.5 parts of jojoba ester (manufactured by International Flora Technologies Ltd., FLORAESTERS 70HG) were mixed as the wax, 1 part of mixture of Red No. 226 and mica was mixed as the coloring agent, 20 parts of mixture of synthetic phlogopite and titanium oxide (manufactured by NIHON KOKEN KOGYO CO., LTD., TWINCLEPEARL (registered trademark) 400, average particle size 37 μm) and 20 parts of mixture of synthetic phlogopite and titanium oxide (manufactured by NIHON KOKEN KOGYO CO., LTD., TWINCLEPEARL (registered trademark) 500, average particle size 90 μm) were mixed as the luster powder, 2 parts of vaseline, 6 parts of diisostearyl malate, and 2 parts of triethylhexanoine were mixed as the non-volatile liquid oil, and 20 parts of dimethicone (manufactured by Shin-Etsu Chemical Co., Ltd., KF-96A-6cs) were mixed as the non-volatile silicone oil. 30 parts of 99% ethanol as the solvent were added to the base, thereby prepared a test sample. The test sample was evaluated. The conditions and evaluation results of the test sample are given in Table 1.

### Example 2

The test sample was prepared and evaluated in the same manner as Example 1 except that the blending amount of synthetic phlogopite was changed to 30 parts as the powder, and the blending amount of mixture of synthetic phlogopite and titanium oxide (manufactured by NIHON KOKEN KOGYO CO., LTD., TWINCLEPEARL (registered trademark) 400, average particle size 37 μm) was changed to 10 parts and the blending amount of mixture of synthetic phlogopite and titanium oxide (manufactured by NIHON KOKEN KOGYO CO., LTD., TWINCLEPEARL (registered trademark) 500, average particle size 90 μm) was changed to 10 parts as the luster powder. The results are given in Table

### Example 3

The test sample was prepared and evaluated in the same manner as Example I except that the blending amount of synthetic phlogopite was changed to 20 parts as the powder, and the blending amount of mixture of synthetic phlogopite and titanium oxide (manufactured by NIHON KOKEN KOGYO CO., LTD., TWINCLEPEARL (registered trademark) 400, average particle size 37 μm) was changed to 15 parts and the blending amount of mixture of synthetic phlogopite and titanium oxide (manufactured by NIHON KOKEN KOGYO CO., LTD., TWINCLEPEARL (registered trademark) 500, average particle size 90 μm) was changed to 15 parts as the luster powder. The results are given in Table

### Example 4

The test sample was prepared and evaluated in the same manner as Example 1 except that synthetic phlogopite was not added as the powder, and the blending amount of mixture of synthetic phlogopite and titanium oxide (manufactured by NIHON KOKEN KOGYO CO., LTD., TWINCLEPEARL (registered trademark) 400, average particle size 37 μm) was changed to 25 parts and the blending amount of mixture of synthetic phlogopite and titanium oxide (manufactured by NIHON KOKEN KOGYO CO., LTD., TWINCLEPEARL (registered trademark) 500, average particle size 90 μm) was changed to 25 parts as the luster powder. The results are given in Table 1.

### Example 5

The test sample was prepared and evaluated in the same manner as Example 1 except that, instead of jojoba ester, 2.5 parts of sucrose tetrastearate triacetate (manufactured by DKS Co. Ltd., Sugar Wax (registered trademark) A-10E) were mixed as the wax. The results are given in Table 1.

### Example 6

The test sample was prepared and evaluated in the same manner as Example 1 except that, instead of jojoba ester, 2.5 parts of mixture of 92.5% paraffin and 7.5% microcrystalline wax (manufactured by NIKKO RICA CORPORATION, PARMIC 160) were mixed as the wax. The results are given in Table 1.

### Example 7

The test sample was prepared and evaluated in the same manner as Example 1 except that, instead of jojoba ester, 2.5 parts of mixture of 80% polyethylene and 201 microcrystalline wax (manufactured by NIKKO RICA CORPORATION, PA wax) were mixed as the wax. The results are given in Table 1.

### Example 8

The test sample was prepared and evaluated in the same manner as Example I except that the blending amount of synthetic phlogopite was changed to 20 parts as the powder, and the blending amount of dimethicone was changed to 10 parts as the non-volatile silicone oil. The results are given in Table

### Example 9

The test sample was prepared and evaluated in the same manner as Example 1 except that synthetic phlogopite was not added as the powder, and the blending amount of dimethicone was changed to 30 parts as the non-volatile silicone oil. The results are given in Table 1.

### Example 10

The test sample was prepared and evaluated in the same manner as Example 1 except that the blending amount of synthetic phlogopite was changed to 7.5 parts as the powder, and the blending amount of jojoba ester was changed to 5 parts as the wax. The results are given in Table 2.

### Example 11

The test sample was prepared and evaluated in the same manner as Example 10 except that, instead of jojoba ester, 5 parts of sucrose tetrastearate triacetate were mixed as the wax. The results are given in Table 2.

### Example 12

The test sample was prepared and evaluated in the same manner as Example 10 except that, instead of jojoba ester, 5 parts of mixture of 92.5% paraffin and 7.5% microcrystalline wax were mixed as the wax. The results are given in Table 2.

### Example 13

The test sample was prepared and evaluated in the same manner as Example 10 except that, instead of jojoba ester, 5 parts of mixture of 80% polyethylene and 20% microcrystalline wax (manufactured by NIKKO RICA CORPORATION, PA wax) were mixed as the wax. The results are given in Table 2.

### Comparative Example 1

The test sample was prepared and evaluated in the same manner as Example I except that the blending amount of synthetic phlogopite was changed to 30 parts as the powder, and the non-volatile silicone oil was not added (the total amount of the non-volatile liquid oil was less than 15% by mass). The results are given in Table 2.

### Comparative Example 2

The test sample was prepared and evaluated in the same manner as Example 1 except that the solvent was not added, the base was produced in a dry process, and heating at the temperature (about 70° C.) equal to or higher than the melting point of the wax was not performed. The results are given in Table 2.

### Comparative Example 3

The test sample was prepared and evaluated in the same manner as Example 1 except that the blending amount of synthetic phlogopite was changed to 12.5 parts as the powder, the wax was not added, and heating at the temperature (about 70° C.) equal to or higher than the melting point of the wax was not performed. The results are given in Table 2.

TABLE 1

| | EXAMPLE | EXAMPLE | EXAMPLE | EXAMPLE | EXAMPLE |
|---|---|---|---|---|---|
| | 1 | 2 | 3 | 4 | 5 |
| TALC | 2.5 | 2.5 | 2.5 | 2.5 | 2.5 |
| SYNTHETIC | 10 | 30 | 20 | — | 10 |
| PHLOGOPITE | | | | | |
| ZINC OXIDE | 1 | 1 | 1 | 1 | 1 |
| MAGNESIUM | 1 | 1 | 1 | 1 | 1 |
| MYRISTATE | | | | | |
| (VINYL | 4 | 4 | 4 | 4 | 4 |
| DIMETHICONE/ | | | | | |
| METHICONE | | | | | |
| SILSESQUIOXANE | | | | | |
| CROSSPOLYMER | | | | | |
| BORON NITRIDE | 8 | 8 | 8 | 8 | 8 |
| JOJOBA ESTER *1 | 2.5 | 2.5 | 2.5 | 2.5 | — |
| SUCROSE | — | — | — | — | 2.5 |
| TETRASTEARATE | | | | | |
| TRIACETATE *2 | | | | | |
| MIXTURE | — | — | — | — | — |
| OF PARAFFIN/ | | | | | |
| MICROCRYSTALLINE | | | | | |
| WAX *3 | | | | | |
| MIXTURE OF | — | — | — | — | — |
| POLYETHYLENE/ | | | | | |
| MICROCRYSTALLINE | | | | | |
| WAX *4 | | | | | |
| MIXTURE OF RED | 1 | 1 | 1 | 1 | 1 |
| NO. 226/MICA | | | | | |
| MIXTURE OF | 20 | 10 | 15 | 25 | 20 |
| SYNTHETIC | | | | | |
| PHLOGOPITE/ | | | | | |
| TITANIUM | | | | | |
| OXIDE *5 | | | | | |
| MIXTURE OF | 20 | 10 | 15 | 25 | 20 |
| SYNTHETIC | | | | | |
| PHLOGOPITE/ | | | | | |
| TITANIUM | | | | | |
| OXIDE *6 | | | | | |
| VASELINE | 2 | 2 | 2 | 2 | 2 |
| DIISOSTEARYL | 6 | 6 | 6 | 6 | 6 |
| MALATE | | | | | |
| TRIETHYLHEXANOINE | 2 | 2 | 2 | 2 | 2 |
| DIMETHICONE | 20 | 20 | 20 | 20 | 20 |
| TOTAL (%) | 100 | 100 | 100 | 100 | 100 |
| PEARL AMOUNT | 40 | 20 | 30 | 50 | 40 |
| KNEADING | WET | WET | WET | WET | WET |
| CONDITIONS | PROCESS | PROCESS | PROCESS | PROCESS | PROCESS |
| IMPACT RESISTANCE | 9 | 10 | 10 | 9 | 7 |
| MOIST FEELING | 4 | 4 | 4 | 4 | 4 |
| GLOSS | 4 | 4 | 4 | 4 | 4 |
| SOFTNESS | 4 | 4 | 4 | 4 | 5 |
| PEARLY FEELING | 4 | 2 | 3 | 5 | 4 |
| EASINESS TO TAKE | 4 | 4 | 4 | 4 | 2 |
| | | EXAMPLE | EXAMPLE | EXAMPLE | EXAMPLE |
| | | 6 | 7 | 8 | 9 |
| | TALC | 2.5 | 2.5 | 2.5 | 2.5 |
| | SYNTHETIC | 10 | 10 | 20 | — |
| | PHLOGOPITE | | | | |
| | ZINC OXIDE | 1 | 1 | 1 | 1 |
| | MAGNESIUM | 1 | 1 | 1 | 1 |
| | MYRISTATE | | | | |
| | (VINYL | 4 | 4 | 4 | 4 |
| | DIMETHICONE/ | | | | |
| | METHICONE | | | | |
| | SILSESQUIOXANE | | | | |
| | CROSSPOLYMER | | | | |
| | BORON NITRIDE | 8 | 8 | 8 | 8 |
| | JOJOBA ESTER *1 | — | — | 2.5 | 2.5 |
| | SUCROSE | — | — | — | — |
| | TETRASTEARATE | | | | |
| | TRIACETATE *2 | | | | |
| | MIXTURE | 2.5 | — | — | — |
| | OF PARAFFIN/ | | | | |
| | MICROCRYSTALLINE | | | | |
| | WAX *3 | | | | |
| | MIXTURE OF | — | 2.5 | — | — |
| | POLYETHYLENE/ | | | | |
| | MICROCRYSTALLINE | | | | |
| | WAX *4 | | | | |
| | MIXTURE OF RED | 1 | 1 | 1 | 1 |
| | NO. 226/MICA | | | | |
| | MIXTURE OF | 20 | 20 | 20 | 20 |
| | SYNTHETIC | | | | |
| | PHLOGOPITE/ | | | | |
| | TITANIUM | | | | |
| | OXIDE *5 | | | | |
| | MIXTURE OF | 20 | 20 | 20 | 20 |
| | SYNTHETIC | | | | |
| | PHLOGOPITE/ | | | | |
| | TITANIUM | | | | |
| | OXIDE *6 | | | | |
| | VASELINE | 2 | 2 | 2 | 2 |
| | DIISOSTEARYL | 6 | 6 | 6 | 6 |
| | MALATE | | | | |
| | TRIETHYLHEXANOINE | 2 | 2 | 2 | 2 |
| | DIMETHICONE | 20 | 20 | 10 | 30 |
| | TOTAL (%) | 100 | 100 | 100 | 100 |
| | PEARL AMOUNT | 40 | 40 | 40 | 40 |
| | KNEADING | WET | WET | WET | WET |
| | CONDITIONS | PROCESS | PROCESS | PROCESS | PROCESS |
| | IMPACT RESISTANCE | 8 | 9 | 7 | 9 |
| | MOIST FEELING | 3 | 4 | 2 | 5 |
| | GLOSS | 4 | 4 | 2 | 5 |
| | SOFTNESS | 4 | 4 | 2 | 5 |
| | PEARLY FEELING | 4 | 4 | 4 | 4 |
| | EASINESS TO TAKE | 2 | 4 | 2 | 4 |

*1 FLORAESTERS 70 HG (MELTING POINT: 66-70° C.)
*2 SUGAR WAX A-10E (MELTING POINT: 44-50° C.)
*3 PARMIC 160 (MELTING POINT: 66-73° C.)
*4 PA WAX (MELTING POINT: 82-89° C.)
*5 TWINCLEPEARL 400
*6 TWINCLEPEARL 500

TABLE 2

| | | | | | COMPARATIVE | COMPARATIVE | COMPARATIVE |
|---|---|---|---|---|---|---|---|
| | EXAMPLE | EXAMPLE | EXAMPLE | EXAMPLE | EXAMPLE | EXAMPLE | EXAMPLE |
| | 10 | 11 | 12 | 13 | 1 | 2 | 3 |
| TALC | 2.5 | 2.5 | 2.5 | 2.5 | 2.5 | 2.5 | 2.5 |
| SYNTHETIC | 7.5 | 7.5 | 7.5 | 7.5 | 30 | 10 | 12.5 |
| PHLOGOPITE | | | | | | | |
| ZINC OXIDE | 1 | 1 | 1 | 1 | 1 | 1 | 1 |
| MAGNESIUM | 1 | 1 | 1 | 1 | 1 | 1 | 1 |
| MYRISTATE | | | | | | | |
| (VINYL DIMETHICONE/ | 4 | 4 | 4 | 4 | 4 | 4 | 4 |
| METHICONE | | | | | | | |
| SILSESQUIOXANE | | | | | | | |
| CROSSPOLYMER | | | | | | | |
| BORON NITRIDE | 8 | 8 | 8 | 8 | 8 | 8 | 8 |
| JOJOBA ESTER *1 | 5 | — | — | — | 2.5 | 2.5 | — |
| SUCROSE | — | 5 | — | — | — | — | — |
| TETRASTEARATE | | | | | | | |
| TRIACETATE *2 | | | | | | | |
| MIXTURE OF PARAFFIN/ | — | — | 5 | — | — | — | — |
| MICROCRYSTALLINE | | | | | | | |
| WAX *3 | | | | | | | |
| MIXTURE OF | — | — | — | 5 | — | — | — |
| POLYETHYLENE/ | | | | | | | |
| MICROCRYSTALLINE | | | | | | | |
| WAX *4 | | | | | | | |
| MIXTURE OF RED | 1 | 1 | 1 | 1 | 1 | 1 | 1 |
| NO. 226/MICA | | | | | | | |
| MIXTURE OF | 20 | 20 | 20 | 20 | 20 | 20 | 20 |
| SYNTHETIC | | | | | | | |
| PHLOGOPITE/ | | | | | | | |
| TITANIUM | | | | | | | |
| OXIDE *5 | | | | | | | |
| MIXTURE OF | 20 | 20 | 20 | 20 | 20 | 20 | 20 |
| SYNTHETIC | | | | | | | |
| PHLOGOPITE/ | | | | | | | |
| TITANIUM | | | | | | | |
| OXIDE *6 | | | | | | | |
| VASELINE | 2 | 2 | 2 | 2 | 2 | 2 | 2 |
| DIISOSTEARYL | 6 | 6 | 6 | 6 | 6 | 6 | 6 |
| MALATE | | | | | | | |
| TRIETHYLHEXANOINE | 2 | 2 | 2 | 2 | 2 | 2 | 2 |
| DIMETHICONE | 20 | 20 | 20 | 20 | — | 20 | 20 |
| TOTAL (%) | 100 | 100 | 100 | 100 | 100 | 100 | 100 |
| PEARL AMOUNT | 40 | 40 | 40 | 40 | 40 | 40 | 40 |
| KNEADING | WET | WET | WET | WET | WET | WET | WET |
| CONDITIONS | PROCESS | PROCESS | PROCESS | PROCESS | PROCESS | PROCESS | PROCESS |
| IMPACT RESISTANCE | 9 | 9 | 8 | — | 5 | 3 | 3 |
| MOIST FEELING | 4 | 4 | 4 | 4 | 1 | 2 | 4 |
| GLOSS | 4 | 4 | 4 | 4 | 1 | 2 | 3 |
| SOFTNESS | 2 | 5 | 4 | 2 | 1 | 3 | 5 |
| PEARLY FEELING | 4 | 4 | 4 | 4 | 3 | 4 | 4 |
| EASINESS TO TAKE | 3 | 3 | 4 | 4 | 1 | 4 | 1 |

*1 FLORAESTERS 70 HG (MELTING POINT: 66-70° C.)
*2 SUGAR WAX A-10E (MELTING POINT: 44-50° C.)
*3 PARMIC 160 (MELTING POINT: 66-73° C.)
*4 PA WAX (MELTING POINT: 82-89° C.)
*5 TWINCLEPEARL 400
*6 TWINCLEPEARL 500

According to Tables 1 and 2, impact resistance, a moist feeling, gloss, softness, pearlescence, and easiness to take were all excellent in the powder solid compositions (Examples 1 to 13) that were obtained by mixing, in a wet process, 15% by mass or more and 40% by mass or less of the non-volatile liquid oil containing the non-volatile silicone oil, 2% by mass or more and 5% by mass or less of the wax in a powder form with a melting point of 55° C. or more and 90° C. or less, and 10% by mass or more and 60% by mass or less of the luster powder with an average particle size of 30 μm or more, volatilizing the solvent at about 50° C. (the temperature at which the wax does not dissolve), and then heating the base at about 70° C. (the temperature at which the wax dissolves).

In contrast, according to table 2, a moist feeling, gloss, softness, and easiness to take were inferior in the powder solid composition (Comparative Example 1) that includes a total amount of less than 15% by mass of the non-volatile liquid oil and was obtained by mixing in a dry process.

According to Table 2, impact resistance was inferior in the powder solid composition (Comparative Example 2) that was obtained by mixing in a dry process and was not heated at a temperature equal to or higher than the melting point of the wax.

According to Table 2, impact resistance and easiness to take were inferior in the powder solid composition (Comparative Example 3) that does not include the wax.

From these results, it has been found that the method for producing a powder solid composition is capable of improving additional properties without deteriorating a feeling of the powder solid composition when used, the method includes the steps of: blending wax in a powder form into a base containing non-volatile liquid oil; dispersing the base in a solvent; volatilizing the solvent at a first temperature where the wax does not dissolve; and heating the base at a second temperature where the wax dissolves.

While embodiments of the present invention have been described above, the present invention is not limited to the specific embodiments, and various modifications and changes are possible within the scope of the invention as claimed.

The present application claims priority to Japanese Patent Application No. 2019-234724, filed Dec. 25, 2019, with the Japanese Patent Office. The contents of which are incorporated herein by reference in their entirety.

### DESCRIPTION OF THE REFERENCE NUMERAL

10 Henschel mixer
11 Mixing vessel
12 Main unit (motor)
13 Rotating shaft
14 Blade
20 Mold
B Powder
W Wax
L Non-volatile liquid oil
S Solvent
O Non-volatile silicone oil
P Slurry
V Volatile solvent
C Compact (powder solid composition)

1. A method for producing a powder solid composition including a base containing non-volatile liquid oil, the method comprising:
blending wax in a powder form into the base;
dispersing the base in a solvent;
volatilizing the solvent at a first temperature where the wax does not dissolve; and
heating the base at a second temperature where the wax dissolves,
wherein the blending amount of the non-volatile liquid oil is 15% by mass or more and 40% by mass or less with respect to the base.

2. The method for producing the powder solid composition according to claim 1, wherein the first temperature is lower than a melting point of the wax.

3. The method for producing the powder solid composition according to claim 1, wherein the second temperature is equal to or higher than a melting point of the wax.

4. The method for producing the powder solid composition according to claim 1, wherein the second temperature is 55° C. or more and 90° C. or less.

5. The method for producing the powder solid composition according to claim 1, wherein non-volatile silicone oil is blended in the non-volatile liquid oil..

6. The method for producing the powder solid composition according to claim 5, wherein a blending amount of the non-volatile silicone oil is 10% by mass or more and 30% by mass or less with respect to the base.

7. The method for producing the powder solid composition according to claim 1, wherein the melting point of the wax is 55° C. or more and 90° C. or less.

8. The method for producing the powder solid composition according to claim 1, wherein the blending amount of the wax is 0.5% by mass or more and 10% by mass or less with respect to the base.

9. The method for producing the powder solid composition according to claim 1, wherein luster powder is blended in the base.

10. The method for producing the powder solid composition according to claim 9, wherein the blending amount of the luster powder is 10% by mass or more and 60% by mass or less with respect to the base.

11. The method for producing the powder solid composition according to claim 9, wherein an average particle size of the luster powder is 30 μm or more.

12. A powder solid composition obtained by the method for producing the powder solid composition of claim 1.

13. A powder solid composition comprising:
15% by mass or more and 40% by mass or less of non-volatile liquid oil in which non-volatile silicone oil is blended;
2% by mass or more and 10% by mass or less of wax, the melting point of the wax being 55° C. or more and 90° C. or less; and
10% by mass or more and 60% by mass or less of luster powder having an average particle size of 30 μm or more.
